(19) United States
(12) Patent Application Publication
(10) Pub. No.: US 2010/0280438 A1
(43) Pub. Date: Nov. 4, 2010

(54) BIDIRECTIONAL CEREBRAL SPINAL FLUID INFUSION CATHETER WITH COOLING MECHANISM AND METHOD OF USE

(75) Inventor: Jeffrey E. Thomas, Hillsborough, CA (US)

Correspondence Address: BENESCH, FRIEDLANDER, COPLAN & ARONOFF LLP;ATTN: IP DEPARTMENT DOCKET CLERK, 200 PUBLIC SQUARE, SUITE 2300, CLEVELAND, OH, 44114-2378, US

(73) Assignee: Dr. Jeffrey E. Thomas, Hillsborough, CA (US)

(21) Appl. No.: 12/431,889

(22) Filed: Apr. 29, 2009

(51) Int. Cl. A61F 7/12; A61M 37/00

(52) U.S. Cl. 604/28; 607/113; 604/43

(57) ABSTRACT

A device and method for delivering a treatment solution includes a bi-directional catheter comprising at least a first lumen and a second lumen, wherein the first lumen comprises a proximal end having an inflow portal and a distal end having an outflow portal, and the second lumen comprises a side wall having at least one inflow portal allowing for fluid communication from an outer surface of the side wall to an inner surface of the side wall and the second lumen further comprises a proximal end having an outflow portal. The device and method further include a pump having an input channel, a reservoir for receiving and containing the treatment solution, a cooling apparatus for cooling the treatment solution, and an output channel. The outflow portal of the second lumen of the catheter is in fluid communication with the input channel of the pump and the output channel of the pump is in fluid communication with the inflow portal of the first lumen.

### BACKGROUND OF THE INVENTION

This application relates in general to an apparatus for treating damage to the central nervous system. Specifically, this application relates to a bidirectional cerebral spinal fluid infusion catheter with cooling mechanism and method of use.

Hypothermia is well established as a neuroprotective strategy for brain injury (stroke, trauma, malignant edema). Currently hypothermia is administered systemically. This intervention is associated with significant medical complications, among them bleeding, pulmonary infection, and the need for sedation with mechanical ventilation, and, often, pharmacologic paralysis.

No effective treatment for damage to the central nervous system, such as completed infarction, hemorrhage or trauma, exists and any maneuver mitigating the catastrophic effect of such damage would be advantageous. This is particularly true for damage to the spinal cord, which renders a human immobile and for which there is no specific remedy.

### DESCRIPTION OF THE DRAWINGS

The accompanying drawings, which are incorporated in and constitute a part of the specification, illustrate various example systems, methods, and so on, that illustrate various example embodiments of aspects of the invention. It will be appreciated that the illustrated element boundaries (e.g., boxes, groups of boxes, or other shapes) in the figures represent one example of the boundaries. One of ordinary skill in the art will appreciate that one element may be designed as multiple elements or that multiple elements may be designed as one element. An element shown as an internal component of another element may be implemented as an external component and vice versa. Furthermore, elements may not be drawn to scale.

FIG. 1 is a cross sectional view of the central nervous system, head, and spine.

FIG. 2 is a schematic representation of a lumbar administration route in a patient.

FIG. 3 is a cross sectional view of a bi-directional dual lumen catheter.

FIG. 4 is a side view of a bi-directional dual lumen catheter and a cooling device.

### DETAILED DESCRIPTION

Naturally-occurring cerebrospinal fluid (CSF) is a clear liquid that occupies the subarachnoid space and the ventricular system around the inside of the brain and the intrathecal space around the spinal cord. CSF acts as a cushion or buffer for the cortex, providing a basic mechanical and immunological protection to the brain inside the skull. CSF also functions to bring nutrients to the brain and spinal cord.

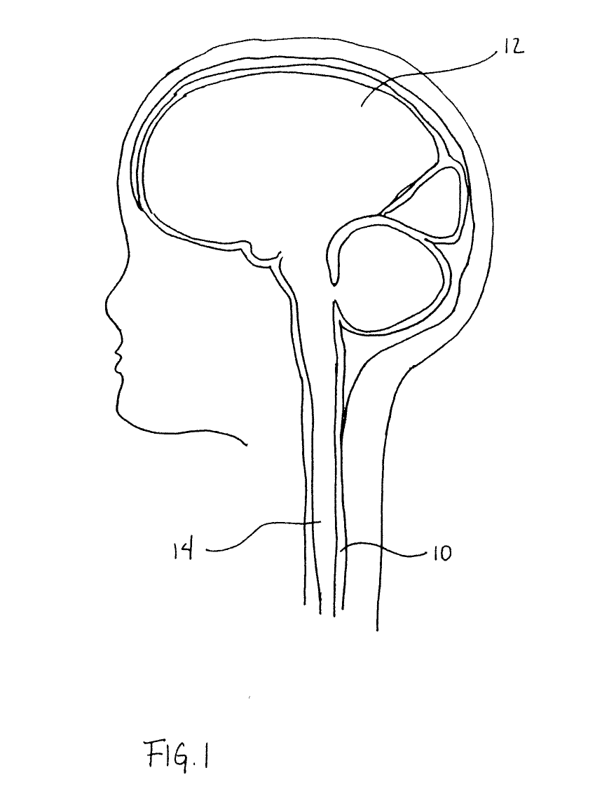

As shown in FIG. 1, CSF 10 may be an ideal carrier for neuroprotective agents and other such neurological treatments because, unlike blood, it directly contacts and circulates around the tissues of the brain 12, the spinal cord 14, and the blood vessels surrounding the brain. One embodiment includes preparing a treatment solution for a patient by cooling the patient's own or administering cooled synthetically derived CSF to a patient for accurate and site specific cooling of the central nervous system, thereby providing neuroprotection.

Neuroprotection may be helpful for patients who have recently undergone a stroke, potential cerebral or spinal ischemia, trauma to the spinal cord or brain, subarachnoid hemorrhage, intracerebral hemorrhage, brain tumor, or any situation where central nervous system damage, cerebral swelling or spinal cord swelling is manifest or expected. Continuously recirculating cooled treatment solution may also be used to prevent tissue ischemia and stroke, as chemotherapy for central nervous system malignancy, and to transport antibiotics for central nervous system infection. Moreover, it is contemplated that such a device may be used for therapeutic irrigation of cerebrospinal fluid, as for example in sever leptomeningeal infection or subarachnoid hemorrhage.

Site specific cooling is more protective than nonspecific cooling of the brain and spinal cord through the cooling of a different compartment, such as the vascular compartment. Currently neuroprotective cooling is done by cooling the vascular compartment and is therefore systemic. Cooling of the head alone is indirect by cooling of the blood first and is impractical. Furthermore, it may not be possible to cool the central nervous system tissue of the spinal cord by indirect methods.

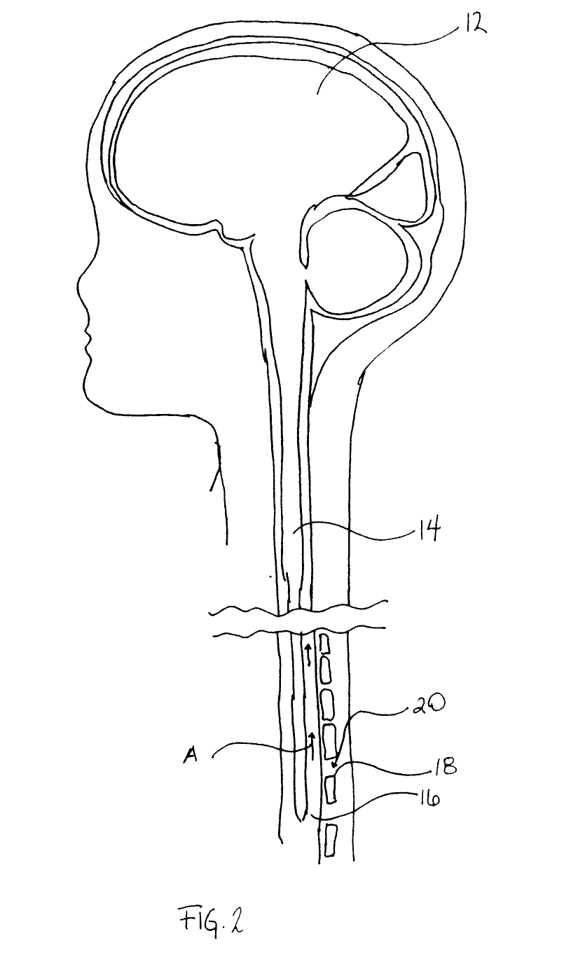

Disclosed herein is a device that would allow a physician to continuously cool the treatment solution, such as the patient's own CSF or a medicated/synthetic CSF solution, by circulating it from the patient's body through a cooling device and returning it to the patient. As shown in FIG. 2, the treatment solution can be administered to an intrathecal space 16 of a patient through a lumbar access point 20 between the vertebrae 18. The treatment solution can then travel throughout the central nervous system such as by the route indicated by arrow A and circulate around the spinal cord 14 and brain tissue 12. Access to the intrathecal space is often attempted below the L1 lumbar vertebrae level to minimize the risk of direct damage to the spinal cord by the needle. It should be recognized that the treatment solution may be administered to a brain or spinal tissue using any known method of accessing the CSF of a patient including, but not limited to, lumbar access to the subarachnoid space, ventriculostomy, or by needle access to the cisterna magna.

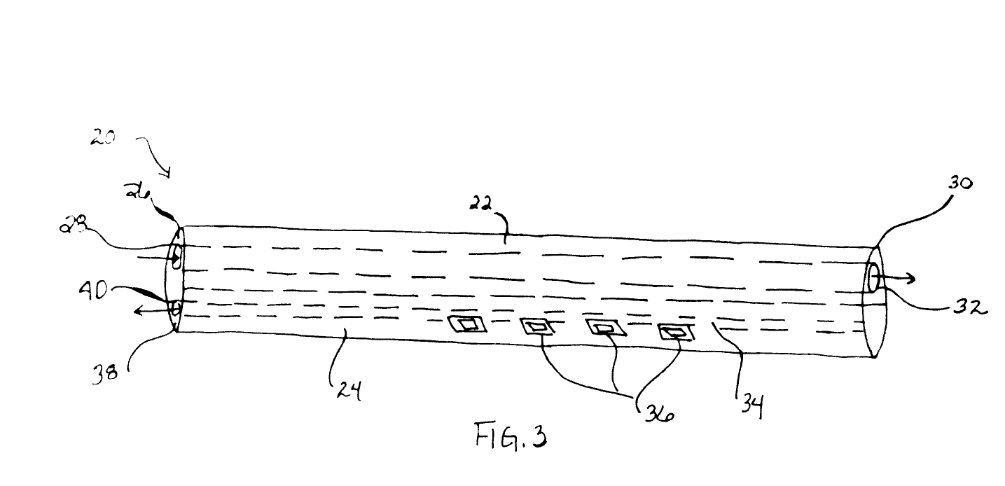

One embodiment of a device for delivering such treatment solution to the central nervous system of a patient includes a bi-directional catheter having at least a first lumen and a second lumen and a pump having an input channel, a reservoir for receiving and containing a treatment solution, a cooling apparatus for cooling the treatment solution, and an output channel. As shown in FIG. 3, the catheter 20 is an elongated tubular member that includes a first lumen 22 and a second lumen 24. The first lumen 22 includes a proximal end 26 with an inflow portal 28 and a distal end 30 having an outflow portal 32. The second lumen 24 includes a sidewall 34 with at least one inflow portal 36 allowing for fluid communication from outside the side wall to inside the side wall. Note that while FIG. 3 illustrates four inflow portals 36, it is specifically contemplated that only one inflow portal may be present in certain embodiments. The second lumen 24 also includes a proximal end 38 and an outflow portal 40. Notably, the distal end of the second lumen is preferably closed to fluid communication. The at least one inflow portal 36 is desirably placed a sufficient distance away, along the horizontal axis, from the outflow portal 32 (located in the distal end 30 of the first lumen 22) to prevent the cooled treatment solution that is discharged from the first lumen 22 from being immediately recirculated into the second lumen 24. Preferably, the fluid pumped through the at least one inflow portal 36 is the patient's own, pre-cooled, CSF.

In one embodiment, the catheter 20 is about 170 mm to about 210 mm in length and from about 1 mm to about 4 mm in diameter, although any catheter of a size and shape that can be inserted into the intrathecal space of a patient without causing damage to the surrounding tissue is contemplated. The catheter 20 may be composed of any suitable material, such as various polymers or plastics. The catheter may also optionally include a tip (not shown) for easily inserting the catheter into the patient's intrathecal space.

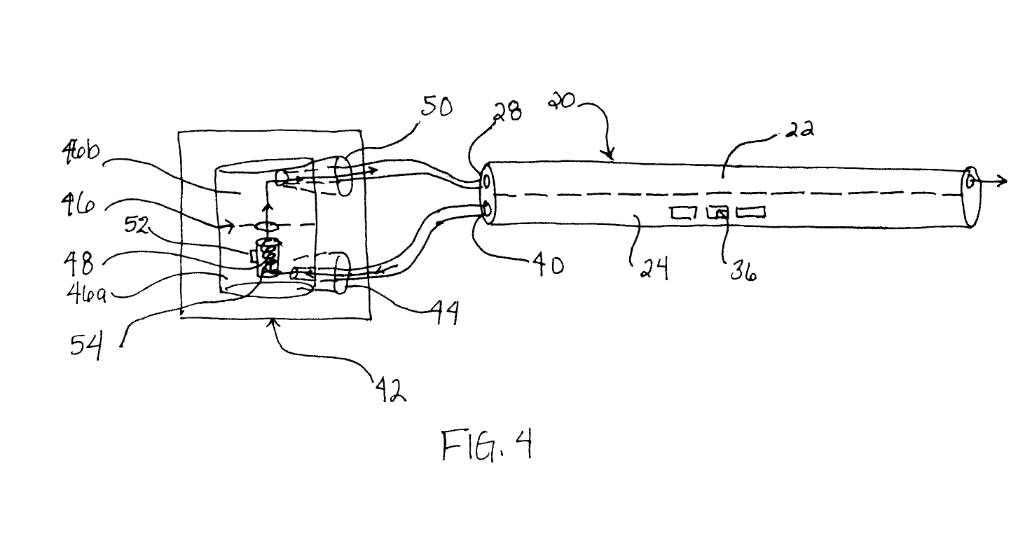

Referring now to FIG. 4, the proximal end of the catheter 20 is attached to a pump 42 for continuously cooling and delivering the treatment solution to a patient. The pump 42 is designed to re-circulate the treatment solution by withdrawing and pumping forward a predetermined volume of solution from and into the patient's body, maintaining the volume of fluid in the patient's system constant over time. Various types of pumps are suitable for use with the catheters disclosed herein, including, but not limited to, an infusion pump. The rate of recirculation may be adjusted over a wide range, but is generally calculated to maintain the patient's own internal pressure and circulation rate.

Specifically, the pump 42 includes an input channel 44, a reservoir 46 for receiving and containing the treatment solution, a cooling apparatus 48 for cooling the treatment solution, and an output channel 50. The pump 42 may be connected to the catheter 20 by providing a pathway for fluid communication between the outflow portal 40 of the second lumen 24 and the input channel 44 of the pump 42 and a pathway for fluid communication between the output channel 50 of the pump 42 and the inflow portal 28 of the first lumen 22. Such pathways may be provided in various ways such as by attaching tubing between the respective openings (as shown) or by directly connecting the pump to the appropriate catheter openings. By connecting the pump directly to the catheter, the system remains closed and sterile for the duration of the patient's treatment.

The pump 42, which may optionally include an integrated pumping mechanism, is designed to withdraw a specific amount of the patient's own CSF from the intrathecal space by drawing it through the inflow portals of the second lumen of the catheter. The CSF may then be transferred to the reservoir of the pump 46 and cooled by the cooling apparatus 48, creating a treatment solution. After cooled, the treatment solution is passed through the outflow channel 50 of the pump 42 to the first lumen of the catheter 20 where it is expelled through the distal end 30 of the first lumen 22 into the patient's body.

Alternatively, a pre-cooled synthetic treatment solution may be present in the reservoir 46 and may be pumped into the patient's intrathecal space as the naturally occurring CSF is pumped into the reservoir 46. The reservoir 46 may be configured in various ways. For example, the reservoir may be one compartment in the pump 42 or it may be separated into a receiving compartment 46a and a delivery compartment 46b. In the reservoir 46, the naturally occurring CSF may optionally be mixed with a medicated treatment solution and re-circulated to the patient via the first lumen 22 of the catheter 20, keeping the patient's level of fluid surrounding the spinal column and brain constant.

In another embodiment, fresh treatment fluid (e.g., synthetic CSF) may be supplied to the reservoir 46 from an outside supply while the patient's naturally occurring CSF is discarded from the system. In such an embodiment an amount of fresh treatment fluid is utilized that is sufficient to keep the level of fluid around the spinal cord and the brain of the patient constant.

The cooling apparatus 48 is capable of producing a cooled treatment solution. In certain embodiments, the cooling apparatus 48 may designed to cool the incoming fluid from the second lumen 24 of the catheter 20 to a temperature of about 15° C. to about 37° C. When the cooled fluid is recirculated to the patient's central nervous system, the system is then cooled by convection or fluid-fluid heat transfer. The cooling apparatus 48 may be configured in various ways. For example, the cooling apparatus 48 may be integrated into the pump 42 or may be a separate device working with the pump 42 to cool the fluid. The cooling apparatus 48 may optionally have a thermostat 52, or a temperature regulation system, that regulates the temperature of the treatment solution as it is cooled. By the phrase “regulates the temperature of the treatment solution as it is cooled” it is meant that the temperature of the treatment solution is controlled or monitored to ensure that the desired temperature of the treatment solution is reached or maintained by the cooling apparatus. When present, the thermostat 52 measures the temperature of the incoming fluid from the patient's body (which enters the pump via the second lumen 24). Alternatively, a juxtaposed thermostat (not shown) may measure the temperature of the outgoing treatment fluid in the reservoir 46 before it leaves via the output channel 50.

The cooling apparatus may optionally include a sealed cooling coil 54 containing a recirculated pressurized liquid refrigerant. Such a cooling coil can be positioned between the input channel 44 and the output channel 50 of the pump 42. In another embodiment, the pump 42 may optionally contain a filtration system, integrated within the fluid circuit, i.e. the closed system that is created by re-circulating the treatment solution through the patient's intrathecal space and the device, to remove any blood or other unwanted contaminants that may be present in the CSF of the spinal cord such as after injury or because of illness. A number of afflictions of the nervous system involve pathological transformation of the subarachnoid space, such as by blood and infection. In the case of subarachnoid hemorrhage, e.g., ruptured cerebral aneurysm or arterio-venous malformation, blood occupies the subarachnoid space around the brain and the spinal cord. Since the subarachnoid space contains the cerebrospinal fluid, surrounding and in contact with the central nervous system at every level, the blood in this space comes to surround the nervous tissue from where it may precipitate further illness, such as cerebral vasospasm. This condition is the primary cause of delayed neurological morbidity and mortality in patients who initially survive ruptured cerebral aneurysm. The condition is associated with subarachnoid hemorrhage, and also to have an incidence that is positively correlated with the volume of subarachnoid hemorrhage. Cerebral vasospasm occurs in a delayed fashion following subarachnoid hemorrhage, and this is believed to be related to a chronic inflammatory reaction that is instigated by the blood. Therefore, the removal of the blood from the subarachnoid space early in the course of illness, i.e., early after subarachnoid hemorrhage would mitigate or prevent cerebral vasospasm.

The described methods and devices provide one such means for removing subarachnoid hemorrhage, by irrigation of the subarachnoid space by a fluid. This fluid may be either the native cerebrospinal fluid of the patient, which is cleansed of blood by a filtration method outside of the body before it is returned to the subarachnoid space, or a synthetic cerebrospinal fluid.

As well, infection of the subarachnoid space can occur, and is alternatively referred to as leptomeningeal infection, meningitis, meningeal infection, ventriculitis, and spinal meningitis. This type of infection consists of growth of microorganisms within the cerebrospinal fluid, and such infections regularly cause death and neurological disability. The subarachnoid space is characterized by a relatively weak immunoresponsiveness and such infections can rapidly progress to overwhelm the patient resulting in death. Therefore, in another embodiment the bidirectional catheters and methods disclosed herein may be used to irrigate or rinse the infected subarachnoid space for the purpose of physically removing microorganisms. The treatment solution used with such catheters and methods may be either the native cerebrospinal fluid of the patient, which has been cleansed of such microorganisms and toxic substances by a filtration method outside of the body before it is returned to the subarachnoid space, or a synthetic cerebrospinal fluid.

Toxic and inflammatory responses to infection within the subarachnoid space and brain are known to occur and may be ameliorated and mitigated by therapeutic cooling of the cerebrospinal fluid. The devices and methods described herein provide a means for such therapeutic cooling by recirculation of the cerebrospinal fluid, with or without the administration of additional synthetic cerebrospinal fluid.

In another embodiment, the devices and methods disclosed herein may be used to treat seizures of the brain, manifested by abnormal spread of electrical activity among neurons of the cortex. These seizures may be a consequence of damage to the central nervous system, or may be spontaneous; and in either case, such seizures may become self-perpetuating, thereby constituting the syndrome of status epilepticus (uninterrupted seizure activity). During the performance of open brain surgery the brain may be arrested with local cooling, usually carried out by topical administration of cold saline solution. Delivery of refrigerated cerebrospinal fluid to the central nervous system by way of the subarachnoid space, as described by the devices and methods disclosed herein, would be advantageous in helping to terminate seizures and the condition of status epilepticus.

### Prophetic Example

### Ischemia

A patient presents to the hospital with signs and symptoms of a stroke in evolution. A CT scan of the brain is performed to demonstrate that there is no brain hemorrhage. The patient's neurological signs demonstrate aphasia and paralysis of the right arm, indicating a stroke in evolution involving the left cerebral hemisphere in the distribution of the middle cerebral artery. The patient is known to have had recent surgery, therefore intravascular thrombolytic agents cannot be used. Instead, the patient undergoes catheterization of his lumbar thecal sac, permitting the administration of a stroke medication that contains properties of site-specific vasodilation effective from the adventitial side of a blood vessel, anti-platelet aggregation, and anti-microvascular sludging. The medication is also delivered by a bidirectional dual lumen catheter, as described herein, coupled with a thermostat-driven recirculation pump with an integrated cooling mechanism.

Because the medication contains no thrombolytic activity, its use is not contraindicated in this patient, and because it is not a thrombolytic, there is no time limitation for its administration to the patient. Because the medication is therapeutically cooled to a desired temperature, e.g., 33° C., it possesses the additional intrinsic property of localized neuronal protection. The delivery of the drug is localized within the central nervous system and therefore, total body cooling is not required. Complications that are usually associated with total body cooling may be obviated by utilizing the devices or methods described herein. As well, the necessity of general anesthesia or sedation may be obviated, and medication can be given to an awake patient, affording the advantage of being able to follow his neurological examination at periodic intervals to measure the effectiveness of treatment. When administration of medication is confined to the cerebrospinal fluid, it provides direct and immediate contact with, and protection of, central nervous tissue at risk. Moreover, when the fluid medium carrying the medication consists of a synthetic cerebrospinal fluid with a density different from a patient's naturally-occurring cerebrospinal fluid, the distribution of such medication can be controlled by tilting the patient, and it may be delivered to the brain from a lumbar intrathecal access site.

In this example, the effects of vasodilation, anti-platelet aggregation, and anti-microvascular sludging are mediated by a mechanism (cyclic GMP activated by nitric oxide) that traverses the blood vessel wall from the cerebrospinal fluid space, and these effects are mitigated by the medication when it is administered in the cerebrospinal fluid. Therefore, the neuroprotective nature of the medication is augmented by local hypothermia of the treated tissue and the treatment solution can still be protective for this patient even if infarction has already occurred.

In such a hypothetical situation, a core of infarction exists, surrounded by a penumbra of damaged but not yet infarcted nervous tissue. Hypothermic protection of this penumbra will, therefore, assist in limiting the damage caused by the initial stroke and its attendant edema, among other mechanisms of secondary brain and spinal cord injury well known to those knowledgeable in the art.

### Prophetic Example

### Spinal Cord Trauma

A patient involved in a motor vehicle accident presents to the Emergency Department of a hospital with acute nonpenetrating trauma to the spinal cord. Neurological examination demonstrates complete paraplegia. Hypothermia protection is considered, but is impractical because it would require intubation, pharmacologic paralysis and induced coma; yet the patient is awake and alert because there is no brain injury. Instead, the patient undergoes catheterization of the thecal sac subarachnoid space with the catheter device disclosed herein, and administration of cooled CSF is initiated, which provides localized hypothermia. Because the patient has had traumatic spine injury, blood exists within the CSF of the spinal cord, and a synthetic CSF solution is simultaneously administered while an integrated filtration system removes red blood cells from the patient's CSF. The removal of the inflammatory influence of this blood, thereby reducing contact with damaged CNS tissue, in conjunction with the hypothermic protection of damaged neurons of the CNS, optimizes recovery from this neurological injury.

### Prophetic Example

### Cerebrovascular Accident with Altered Mental Status

A patient presents to the Emergency Department of a hospital with painless weakness of the right side of his body and speech dysfunction. Neurological examination demonstrates right hemiplegia and aphasia. CT scan of the brain demonstrates low attenuation changes of the left cerebral hemisphere in the distribution of the middle cerebral artery, with cerebral edema. The patent also exhibits confusion and lethargy. The history indicates that the patient had weakness before going to bed the previous evening. Because he is beyond the usual time window for administration of a thrombolytic agent, therapeutic options are limited. Endovascular surgical options are also eliminated by the relatively late presentation. Neuroprotection is now a central feature of available therapeutic options. Because the patient has an altered mental status and is demonstrating neurological deterioration with evidence of early cerebral edema on the CT scan, prudent management is intubation and transfer to Intensive Care Unit, with institution of cerebral protection by any of several available methods, including pentobarbital coma and hypothermia. Disadvantages of pentobarbital coma include infection, disturbance of gut motility with resultant insufficient nutrition, and hypotension. Disadvantages of systemic hypothermia include pulmonary infection and bleeding, from coagulation abnormalities. It is decided that the patient may experience the least risk with site-specific, organ system-specific hypothermia delivered to the CNS. A specially designed lumbar catheter as disclosed herein is used to deliver cooled cerebrospinal fluid. In this situation, the CSF is cooled by convection, and the circulating CSF surrounding the damaged brain tissue is also cooled, although the treatment is delivered via catheter access to the lumbar thecal CSF. Alternatively, the recirculation pump is used to deliver a measure of hyperbaric synthetic CSF, which reaches the intracranial space rapidly through simple tilting of the patient into Trendelenburg position (head down). Alternatively, the hypobaric variation of synthetic CSF is used, in which case the correct patient positioning is reverse-Trendelenburg (head up).

In another variation, CSF cooling may be achieved through ventricular access to the CSF, which requires installation of a ventricular catheter (ventriculostomy). In this situation, a special ventricular catheter is used that has the physical characteristics and properties of the multi-lumen bidirectional flow catheter. In the case of the comatose or obtunded patient, this option may be easily substituted, whereas with the fully awake stroke patient the option may be less desirable, and the lumbar route of administration would be favored.

### Prophetic Example

### Cerebrovascular Accident without Altered Mental Status

A patient presents to the emergency department of a hospital with acute stroke. He has arrived at the hospital approximately 4 hours after the stroke. Therefore, he is ruled out as a candidate for intravenous tPA, and the hospital does not have availability of a neurointerventional specialist for consideration of intraarterial thrombolysis. Examination reveals him to be awake and alert, with paralysis of the left side of his body. CT perfusion scan demonstrates a large area of the right cerebral hemisphere with infarction in the distribution of the right middle cerebral artery, and also a large area of brain tissue with diffusion/perfusion mismatch, indicating that a larger area of brain tissue is at risk to go on to cerebral infarction. Therapeutic options are now limited to optimization of collateral circulation of the brain, and neuroprotection. From the effects of cerebral edema and inflammatory brain reaction, the patient is at risk for neurological deterioration and more extensive infarction within the next several days. Because he is alert, the surgical procedure of decompressive hemicraniectomy is not under immediate consideration. For the same reason, systemic hypothermia with intubation and pharmacologic paralysis are not immediate considerations. The patient would ideally benefit from a localized form of neuroprotection that does not involve cooling of the entire body such as is provided by the devices and methods disclosed herein. The patient may have local anesthesia administered to the skin of the lumbar spine for the purpose of installing the catheter, without the need for general anesthesia.

The patient in this example may also benefit from the lumbar intrathecal administration of a special formula for stroke, which is the subject of currently pending U.S. application Ser. No. 12/412,011, filed on Mar. 26, 2009, and herein incorporated by reference. This medication provides the optimization of available collateral blood vessels, increasing regional cerebral blood flow and local cerebral oxygen tension and may be delivered in refrigerated form to provide additional neuroprotection via hypothermia.

### Prophetic Example

### Brain Trauma, CHT

A patient presents to the emergency department of a hospital, having sustained closed head trauma with subsequent brain injury. The patient's CT scan demonstrates frontal and temporal contusions and generalized brain edema. Intracranial hypertension is diagnosed based on review of CT scan and clinical condition of the patient. In this case, the patient suffers from raised intracranial pressure consequent to brain contusion and edema. This condition can be expected to be aggravated over the next 3-5 days, as cerebral edema and inflammatory brain reaction evolve. The cause of neurological deterioration and death in such cases is frequently the result of this type of secondary brain injury which reliably follows the trauma. A treatment to diminish and mitigate this reaction would be valuable. There are disadvantages to the use of whole-body hypothermia, such as pulmonary infection and bleeding abnormalities, which in themselves could worsen the bleeding contusions in the brain, and other injuries. The selective hypothermia method described herein is, therefore, selected and a lumbar intrathecal access to the cerebrospinal fluid is established with the bidirectional flow catheter and CSF irrigation and cooling system. The central nervous system of the patient is selectively cooled to a desired temperature, between 15 and 37 degrees Celsius, as the cerebrospinal fluid is recirculated by the cooling pump. In an alternative method, synthetic cerebrospinal fluid is used to substitute a portion or the entirety of the patient's native CSF.

In yet another alternative embodiment, the synthetic CSF is hyperbaric and flows rapidly cephalad as the patient is moved into Trendelenburg position, more rapidly reaching the target tissue of the brain and bathing it. The treatment is administered for a time period corresponding to the most severe phase of the illness, e.g., 3-5 days. Since systemic hypothermia is obviated, bleeding complications and infectious complications related to that treatment are avoided, while preserving its neuroprotective benefits.

### Prophetic Example

### Cerebral Hypoperfusion

A patient suffering from severe cerebral ischemia from hypoperfusion secondary to intracranial arterial stenosis is under observation in the intensive care unit. The patient has no neurological deficit, but develops right hemiparesis and aphasia when his mean arterial blood pressure is permitted to fall below 100 mm Hg. Therefore, he is maintained on pressor agents (such as Neosynephrine) and intravascular volume expansion with colloid and crystalloid in order to prevent a fall in blood pressure. His collateral circulation will mature sufficiently within the next several days, to the point that he will no longer be dependent on this management to preserve his brain function. His cardiac function, however, has become marginal within the last 12 hours, and he is exhibiting signs of congestive heart failure. Because of this it has become progressively more difficult to maintain adequate blood pressure. CT perfusion scan is done, demonstrating no infarction but a diffusion/perfusion mismatch that identifies a substantial portion of the left cerebral hemisphere as being at risk for stroke.

Under these circumstances, therapeutic maneuvers to confer neuroprotection are desirable. Hypothermia will slow the metabolism of the target tissue, rendering it less vulnerable to ischemic injury for the time period necessary for the patient to optimize his collateral circulation. In administering localized, site-specific hypothermia by way of the methods and devices described herein, systemic effects and disadvantages of whole-body hypothermia are avoided.

A distinct advantage of localized hypothermia in this situation is the ability to monitor the examination of the patient as the treatment proceeds, which would not be possible were the patient to be systemically cooled, as is done with external cold packs, heat transfer pads and intravascular cooling of the blood. In an alternative embodiment, the cooled cerebrospinal fluid consists of a synthetic fluid substitute with antiplatelet, anti-microvascular sludging and vasodilator properties, all such properties contributing to improved rheology and volume of collateral circulation.

### Prophetic Example

### Cerebral Vasospasm

A patient presents to the hospital with ruptured cerebral aneurysm and subarachnoid hemorrhage (SAH). The clinical grade is moderate (the patient is awake without focal deficit), but the radiographic grade is consistent with a voluminous SAH and, therefore, risk of cerebral vasospasm. His aneurysm is treated by neurosurgical operation within 24 hours, and his recovery in ICU is unremarkable for 6 days. On the 7th day, however, he develops obtundation and hemiparesis. Transcranial Doppler reveals cerebral vasospasm in the right middle cerebral artery, the location of the ruptured and now clipped aneurysm. This is confirmed by CT angiography, and CT perfusion shows a diffusion/perfusion mismatch in a substantial region of the right hemisphere served by the middle cerebral artery.

Symptomatic cerebral vasospasm is therefore established, and substantial cerebral territory at risk of infarction is defined. Although cerebral balloon angioplasty may be used in this situation, it is limited by the requirement for experienced neurointerventional personnel and equipment, as well as by the anatomical distribution of the vasoconstriction (angioplasty can only be performed in the proximal vasculature, whereas the effects of vasospasm are wide-reaching). Intravascular volume expansion and induced hypertension have limited power to alleviate the condition, and may not be usable at all in elderly patients or in patients prone to congestive heart failure or volume overload.

Under these conditions, it is advantageous to confer hypothermic protection upon the brain at risk, and to do so in a manner that does not invite systemic complications such as pulmonary infection and bleeding in this patient with recent SAH. Thus, the patient is treated with lumbar thecal catheterization as described herein, and selective hypothermic brain protection is administered via the cooled cerebrospinal fluid. In an alternative treatment paradigm, the infused cerebrospinal fluid is synthetic, as described elsewhere herein, and contains agents conferring properties of vasodilation, antiplatelet and anti-microvascular sludging, and may also be delivered in a hypobaric or hyperbaric preparation of the synthetic cerebrospinal fluid, for more rapid or accurate delivery to the intracranial subarachnoid space. These properties are valuable to optimize rheology and volume of the cerebral circulation, thereby augmenting it and optimizing the collateral circulation that mitigates cerebral ischemia.

### Prophetic Example

### Leptomeningeal Infection

A patient is admitted to the intensive care unit of the hospital with high fever and delirium. Examination reveals clouded sensorium, neck stiffness with positive clinical signs of meningitis (Kernig's and Brudzinski's signs). The patient's body temperature is 39.5° C. The patient undergoes CT scan of the brain which reveals no evidence of hemorrhage or mass lesion. A lumbar puncture is performed and reveals bacteria in the CSF, with hypoglycorrhachia and elevated neutrophil count. The diagnosis of bacterial meningitis is established and appropriate antibiotics are begun.

Under these circumstances, the practitioner schooled in the art recognizes that irrigation of the CSF to diminish the number of microorganisms, and to remove toxic inflammatory substances, may be valuable. Use of the methods disclosed herein is therefore instituted in the form of a lumbar intrathecal catheter and recirculation cooling pump. The sCSF solution is used to irrigate the subarachnoid space. In an alternative method, the cerebrospinal fluid infusion is cooled to a predetermined temperature, affording a means to selectively cool the central nervous system elements directly in contact with it, and thereby providing localized neuroprotection against harmful effects of inflammation and hyperthermia.

In yet another alternative method, said CSF infusion is delivered as a hyperbaric or hypobaric formulation for more rapid and accurate delivery to the intracranial subarachnoid space. The treatment is delivered in this manner for several days or longer, as long as the threat to central nervous tissue persists. The treatment may also be protective against seizure disorder, known to the practitioner of the art to be commonly associated with severe leptomeningeal infection.

### Prophetic Example

### Subarachnoid Hemorrhage

A patient presents to the hospital with high-grade aneurysmal subarachnoid hemorrhage. The abundance of blood in the subarachnoid space consequent to the hemorrhage, in addition to immediately threatening the patient's life through abruptly increased intracranial pressure, creates other hazards consequent to inflammatory brain response (cerebral edema) and delayed chronic cerebral vasoconstriction (vasospasm). Such inflammatory responses are known to be associated with the blood itself in the subarachnoid space, and removal of said blood, either partially or in its entirety, from the subarachnoid space would be beneficial in mitigating or preventing these complications.

Therefore, the one of the methods disclosed herein is instituted for the purpose of therapeutic irrigation of the cerebrospinal fluid, either substituting the sCSF as blood-filled CSF is simultaneously removed, or alternatively by interposing a filtration device in the CSF conduit. In an alternative embodiment, the infused and recirculated cerebrospinal fluid is administered after cooling to a predetermined temperature in the range of 15 to 37° C. for purposes of neuroprotection.

### Prophetic Example

### Status Epilepticus

A patient is admitted to the hospital with continuous grand mal seizures. The patient is unable to return to a wakeful state before the next seizure occurs, thereby meeting the criterion for status epilepticus and neurological emergency. A prescribed medical regimen for the emergency treatment of this dangerous condition exists, but is not always successful, whereupon general anesthesia is instituted.

Administration of cold saline to the seizing brain may immediately halt seizure, therefore, the therapeutic cooling of the cerebrospinal fluid, in direct and universal contact with the brain and spine, may have a similar beneficial effect in arresting seizures.

In this case, therefore, the failure of the medical regiment to arrest the seizures results in the implementation of the methods disclosed herein, obviating the necessity for general anesthesia. Alternatively, if general anesthesia had been required for circumstantial reasons to arrest the seizures, the implementation of the one of the methods of selective CSF cooling disclosed herein would make it possible to remove general anesthesia from the patient.

While example methods and compositions have been illustrated by describing examples, and while the examples have been described in considerable detail, it is not the intention of the applicants to restrict or in any way limit the scope of the appended claims to such detail. It is, of course, not possible to describe every conceivable combination of components or methodologies for purposes of describing the systems, methods, devices, and so on, described herein. Additional advantages and modifications will readily appear to those skilled in the art. Therefore, the invention is not limited to the specific details, the representative apparatus, and illustrative examples shown and described. Thus, this application is intended to embrace alterations, modifications, and variations that fall within the scope of the appended claims. Furthermore, the preceding description is not meant to limit the scope of the invention. Rather the scope of the invention is to be determined by the appended claims and their equivalents.

1. A device for delivering a treatment solution, the device comprising:
a bi-directional catheter comprising a first lumen and a second lumen,
wherein the first lumen comprises a proximal end having an inflow portal and a distal end having an outflow portal, and
the second lumen comprises a side wall having at least one inflow portal allowing for fluid communication from outside the side wall to inside the side wall and the second lumen further comprising a proximal end having an outflow portal; and
a pump comprising an input channel, a reservoir for receiving and containing the treatment solution, a cooling apparatus for cooling the treatment solution, and an output channel;
wherein the outflow portal of the second lumen of the bi-directional catheter is in fluid communication with the input channel of the pump and the output channel of the pump is in fluid communication with the inflow portal of the first lumen.

2. The device of claim 1, wherein the cooling apparatus comprises an integrated thermostat for regulating temperature of the treatment solution.

3. The device of claim 1, wherein the pump further comprises a thermostat for regulating the temperature of the treatment solution.

4. The device of claim 1, wherein the bi-directional catheter is an intrathecal catheter capable of lumbar administration of the treatment solution to a patient's central nervous system.

5. The device of claim 1, wherein the bi-directional catheter is an intrathecal catheter capable of ventricular administration of the treatment solution to a patient's central nervous system.

6. The device of claim 1, wherein the pump is an infusion pump capable of continuously pumping a desired amount of the treatment solution from the input channel to the output channel.

7. A method of treating or preventing damage to a patient's central nervous system, comprising:
providing a catheter comprising a first lumen and a second lumen,
wherein the first lumen comprises a proximal end having an inflow portal and a distal end having an outflow portal, and
the second lumen comprises a sidewall having at least one inflow portal providing fluid communication from outside the side wall to inside the side wall and the second lumen further comprises a proximal end having an outflow portal;
providing a pump comprising an input channel, a reservoir for receiving and containing the treatment solution, a cooling apparatus for producing a cooled treatment solution, and an output channel;
wherein the outflow portal of the second lumen of the catheter is in fluid communication with the input channel of the pump and the output channel of the pump is in fluid communication with the inflow portal of the first lumen;
inserting the catheter into the patient's intrathecal space;
withdrawing a portion of the patient's cerebrospinal fluid through the second lumen catheter by use of the pump;
delivering the patient's cerebrospinal fluid to the reservoir of the pump and cooling the patient's cerebrospinal fluid with the cooling apparatus to produce a cooled treatment fluid;
introducing the cooled treatment fluid to the patient's intrathecal space through the first lumen of the catheter.

8. The method of claim 7, wherein the pump continuously recirculates the patient's cerebrospinal fluid from the patient's intrathecal space to the reservoir to provide the cooled treatment solution.

9. The method of claim 7, wherein the cooling apparatus comprises an integrated thermostat for regulating temperature of the cooled treatment solution.

10. The method of claim 7, wherein the pump further comprises a thermostat for regulating the temperature of the cooled treatment solution.

11. The method of claim 7, wherein the catheter is an intrathecal catheter capable of lumbar administration of the cooled treatment solution to a patient's central nervous system.

12. The method of claim 7, wherein the catheter is an intrathecal catheter capable of ventricular administration of the cooled treatment solution to a patient's central nervous system.

13. The device of claim 1, wherein the pump is an infusion pump capable of continuously pumping a desired amount of the treatment solution from the input channel to the output channel.

14. A method treating or preventing damage to the central nervous system of a patient, comprising:
providing a catheter comprising a first lumen and a second lumen,
wherein the first lumen comprises a proximal end having an inflow portal and a distal end having an outflow portal, and
the second lumen comprises a sidewall having at least one inflow portal allowing for fluid communication from outside the side wall to inside the side wall and the second lumen further comprising a proximal end having an outflow portal;
providing a pump comprising an input channel, a reservoir for receiving and containing a treatment solution, a cooling apparatus for producing a cooled treatment solution, and an output channel;
wherein the outflow portal of the second lumen of the catheter is in fluid communication with the input channel of the pump and the output channel of the pump is in fluid communication with the inflow portal of the first lumen;
inserting the catheter into the patient's intrathecal space;
withdrawing a portion of the patient's cerebrospinal fluid;
cooling the patient's cerebrospinal fluid to produce the cooled treatment fluid; and
introducing the cooled treatment fluid to the patient's intrathecal space through the first lumen of the catheter.

15. The method of claim 7 or 14, wherein the cooled treatment solution is cooled to about 15° C. to about 37° C.

16. The method of claim 14, wherein the reservoir comprises a receiving compartment and a delivery compartment, and wherein the cooling apparatus receives the patient's cerebrospinal fluid from the receiving compartment and delivers the cooled treatment solution to the delivery compartment.

17. The method of claim 14, wherein the pump continuously recirculates the patient's cerebrospinal fluid from the patient's intrathecal space to the reservoir to provide the cooled treatment solution.

18. The method of claim 14, wherein the cooling apparatus comprises an integrated thermostat for regulating temperature of the cooled treatment solution.

19. The method of claim 14, wherein the pump further comprises a thermostat for regulating the temperature of the cooled treatment solution.
